(19) United States
(12) Patent Application Publication
(10) Pub. No.: US 2022/0401491 A1
(43) Pub. Date: Dec. 22, 2022

(54) MESENCHYMAL STEM CELL EXPRESSING TRAIL AND CD, AND USE THEREOF

(71) Applicant: SLBIGEN INC., Seongnam-si (KR)

(72) Inventors: Young Chul SUNG, Seoul (KR); Soon Min LEE, Seoul (KR); Hey-yon KIM, Seoul (KR)

(73) Assignee: SLBIGEN INC., Seongnam-si (KR)

(21) Appl. No.: 17/853,259

(22) Filed: Jun. 29, 2022

Related U.S. Application Data
(63) Continuation of application No. 16/075,380, filed on Aug. 3, 2018, now abandoned.

(30) Foreign Application Priority Data
Feb. 5, 2016 (KR) 10-2016-0015103

(51) Int. Cl. A61K 35/28; C12N 9/78; C12N 15/86; A61K 48/00; C07K 14/47; C12N 9/10; A61P 35/00; A61K 38/17; A61K 38/19

(52) U.S. Cl. CPC A61K 35/28; C12N 9/78; C12N 15/86; A61K 48/00; C07K 14/47; C12N 9/10; A61P 35/00; A61K 38/1761; A61K 38/191; C12Y 305/04001

(57) ABSTRACT

A recombinant lentiviral vector containing a gene encoding a TRAIL protein and a CD protein; and a cell that is transfected with the lentivirus produced by using the vector. A host cell transfected with the recombinant lentivirus maintains a high cell proliferation rate and overexpresses a TRAIL protein and a CD protein. Thus, a mesenchymal stem cell transfected with the lentivirus may be usefully employed as a cell therapeutic agent.

### CROSS REFERENCE TO RELATED APPLICATIONS

This application is a Continuation of U.S. application Ser. No. 16/075,380 filed August 3, 2018, which is National Stage of International Application No. PCT/KR2017/001308 filed Feb. 6, 2017, claiming priority based on Korean Patent Application No. 10-2016-0015103 filed Feb. 5, 2016.

### SeEQUENCE LISTING

The content of the electronically submitted sequence listing, file name: Sequence Listing As Filed.txt; size: 40,596 bytes; and date of creation: Jun. 29, 2022, filed herewith, is incorporated herein by reference in its entirety.

### TECHNICAL FIELD

The present invention relates to a recombinant lentiviral vector comprising a gene encoding a TRAIL (TNF-related apoptosis-inducing ligand) protein and a CD (cytosine deaminase) protein, and a cell transfected with the lentivirus produced by using the vector.

### BACKGROUND ART

Therapies utilizing cells are being developed globally, and especially, the stem cell therapy market is showing a steady upward trend with an annual average growth rate of 11.7%.

Mesenchymal stem cells (MSCs), which are adult stem cells, are multipotent cells that can differentiate into bones, cartilages, muscles, fats, and fibroblasts, etc. In addition, MSCs can be obtained from various adult tissues such as bone marrow, umbilical cord blood, and fats, relatively easily. MSCs are characterized by their ability to migrate to the site of inflammation or injury, which is also a great advantage as a delivery vehicle for delivering a therapeutic drug. Further, the immune function of the human body can be regulated by inhibiting the functions of immune cells such as T cells, B cells, dendritic cells and natural killer cells. Additionally, MSCs have an advantage that it can be cultured relatively easily in vitro, and thus studies for using MSCs as a cell therapeutic agent are being actively carried out.

However, despite such advantages of MSCs, there are following problems in producing MSCs that can be used clinically as a cell therapeutic agent. First, since there is a limitation in the proliferation of MSCs, it is difficult to produce them in large quantities. Second, since the MSCs obtained are heterogenous, it is difficult to maintain the same effect in every production. Third, the use of MSCs alone is not therapeutically effective.

On the other hand, Korean Patent No. 1585032 discloses a cell therapeutic agent containing mesenchymal stem cells cultured in a hydrogel. The above document provides a composition that can be administered directly by shortening the pretreatment process in the step of isolating mesenchymal stem cells for use as a cell therapeutic agent. However, the problems of the mesenchymal stem cells described above and the method for solving the problems are not mentioned at all. Therefore, it is necessary to study mesenchymal stem cells which can be useful as a cell therapeutic agent.

### DISCLOSURE OF INVENTION

### Technical Problem

An object of the present invention is to provide a recombinant lentivirus comprising a gene encoding a TRAIL protein and a CD protein and a host cell transfected with the above recombinant lentivirus.

Another object of the present invention is to provide a pharmaceutical composition comprising the above recombinant lentivirus or host cell.

### Solution to Problem

In accordance with one object of the present invention, there is provided a recombinant lentiviral vector comprising a gene encoding a TRAIL protein and a CD protein.

Further, in accordance with another object of the present invention, there is provided a recombinant lentivirus comprising a gene encoding a TRAIL protein and a CD protein.

Further, in accordance with another object of the present invention, there is provided a host cell transfected with the above recombinant lentivirus.

Further, in accordance with another object of the present invention, there is provided a pharmaceutical composition for preventing or treating cancer comprising the above recombinant lentivirus as an active ingredient.

Further, in accordance with another object of the present invention, there is provided a pharmaceutical composition for preventing or treating cancer comprising the above host cell as an active ingredient.

### Advantageous Effects of Invention

A host cell transfected with a recombinant lentivirus comprising a gene encoding a TRAIL protein and a CD protein of the present invention expresses a TRAIL protein and a CD protein and maintains a high cell proliferation rate. In addition, abnormal differentiation can be inhibited and the possibility of tumor formation can be blocked, indicating high safety. Therefore, the lentivirus or the host cell can be useful as a cell therapeutic agent.

### BRIEF DESCRIPTION OF DRAWINGS

FIG. 1 is a graph comparing cell proliferation rates of immortalized MSCs and non-immortalized MSCs:

imMSC: immortalized MSC;

MSC: non-immortalized MSC;

X axis: incubation period; and

Y axis: cumulative population doubling level (PDL).

FIG. 2 is a schematic representation of the structure of a gene construct inserted into a pBD-4 lentiviral vector:

TRE: a promoter comprising tetracycline response elements;

TRAIL: TNF-associated apoptosis-inducing ligand;

IRES: internal ribosome entry site; and

CD: CD protein.

FIG. 3 compares the expressions of the surface antigen proteins CD90, CD44, CD105, CD73, etc., which are inherent characteristics of MSC, of the MSC after gene manipulation of the BM-03 cell line and of the bone marrow-derived MSC.

FIG. 4 shows the differentiation ability of the BM-03 cell line after gene manipulation, in which adipogenesis, osteogenesis, and chondrogenesis of the BM-03 cell line after gene manipulation were identified.

FIG. 5 shows the results of detection for transgene(TRAIL and CD) in BM-03, a deposited strain. Lane 1 shows a marker, lane 2 shows a negative control, lane 3 shows a positive control, and lanes 4 to 6 show BM-03.

FIG. 6 shows doxycycline dependent TRAIL expression using FACS.

FIG. 7 shows cell death by treating mesenchymal stem cells expressing a TRAIL protein and a CD protein with SFC, which was examined by FACS.

FIG. 8 is a graph showing the PDL of BM-03 cells obtained by subculture.

FIG. 9 shows the results of analysis of the karyotype of the BM-03 cell into which the gene was introduced.

### BEST MODE FOR CARRYING OUT THE INVENTION

Hereinafter, the present invention will be described in detail.

The present invention provides a recombinant lentiviral vector comprising a gene encoding a TRAIL protein and a CD protein.

As used herein, the term “TNF-related apoptosis-inducing ligand (hereinafter, referred to as TRAIL)” protein belongs to the type 2 transmembrane cytokine in the TNF family. The TRAIL selectively induces apoptosis of transformed cells as one of suicide genes. Specifically, TRAIL binds to death receptor-4 (DR-4), DR-5, decoy receptor or decoy receptor-2 present on the cell surface to activate the apoptosis signal transduction system. TRAIL is not toxic to normal cells, and is known to specifically induce apoptosis of cancer cells only.

A TRAIL protein according to the present invention may be a human-derived protein. The TRAIL protein is present in the form of a homotrimer capable of binding to three receptors. The TRAIL protein of the present invention may be a polypeptide having the amino acid sequence of SEQ ID NO: 1. The TRAIL protein may have about 70%, 80%, 90%, 95% or higher homology with the amino acid sequence of SEQ ID NO: 1. Meanwhile, the gene encoding the TRAIL protein may be a polynucleotide having the nucleotide sequence of SEQ ID NO: 2. In addition, the nucleotide sequence encoding the TRAIL protein may have about 70%, 80%, 90%, 95% or higher homology with the nucleotide sequence of SEQ ID NO: 2.

As used herein, the term “CD protein” is a cytosine deaminase protein, and may be in the form of a fusion protein in which CD and uracil phosphoribosyltransferase (UPRT) are linked, and the term “CD protein” as used herein, can be used interchangeably with “CD::UPRT”.

The apoptosis of a cell expressing the above CD protein is induced by converting 5-fluorocytosine (5FC) into 5-fluorouracil (5FU) having strong cytotoxicity. Therefore, the apoptosis of the cell containing a lentiviral vector comprising a CD protein gene can be induced by treatment with 5FC.

The sequence of the gene encoding a CD protein according to the present invention is a codon-optimized sequence by fusing the FCY1 gene encoding the CDase of charomyces cerevisiae and the FUR1Δ105 gene encoding the UPRTase in which 35 amino acids from the N-terminal are deleted. Such sequences may be those described in U.S. Pat. No. 5,338,678, International Patent Publication No. WO 96/16183, and International Patent Publication No. WO 99/54481. According to one embodiment, the CD protein may be a polypeptide having the amino acid sequence of SEQ ID NO: 3. In addition, the CD protein may have about 70%, 80%, 90% or 95% or higher homology with the amino acid sequence of SEQ ID NO: 3. Meanwhile, the gene encoding the CD protein may be a polynucleotide having the nucleotide sequence of SEQ ID NO: 4. In addition, the nucleotide sequence encoding the CD protein may have about 70%, 80%, 90% or 95% or higher homology with the nucleotide sequence of SEQ ID NO: 4.

The term “lentiviral vector” as used herein is a kind of retroviruses, which is a vector in the form of single stranded RNA, and may also be referred to as “lentivirus transfer vector”. The lentiviral vector can be inserted into the genomic DNA of a target cell of infection to stably express the gene, and can transfer the gene to the mitotic and non-mitotic cells. Since the vector does not induce the immune response of a human body, its expression is continuous. In addition, there is an advantage that genes of a large size can be delivered as compared to an adenovirus vector which is a conventional virus vector.

The lentiviral vector may further comprise a gene encoding a thymidine kinase (TK) protein. The TK protein is an enzyme that catalyzes the thymidyl acid production reaction by binding phosphoric acid at the y-position of ATP to thymidine, whereby thymidine is transformed into a triphosphate form. The modified thymidine cannot be used for DNA replication, and is known to induce death of cells containing it. The TK protein for use herein may be one of any known sequences. According to one embodiment, the TK protein may be a polypeptide having the amino acid sequence of SEQ ID NO: 5. Meanwhile, the gene encoding the TK protein may be a polynucleotide having the nucleotide sequence of SEQ ID NO: 6.

The recombinant lentiviral vector of the present invention can comprise 1 or 2 promoters. The promoter may be a cytomegalovirus (CMV), respiratory syncytial virus (RSV), human elongation factor-1 alpha (EF-1a) or tetracycline response elements (TRE) promoter. According to one embodiment, the recombinant lentiviral vector can regulate the expression of a TRAIL or a CD protein by one promoter. The promoter can be operably linked to a gene encoding a protein to be expressed.

According to one embodiment, the TRAIL and CD proteins may be linked to a TRE promoter. The TRE promoter can activate the transcription of the gene linked to the promoter by the tetracycline transactivator (tTA) protein. Specifically, the tTA protein binds to the TRE promoter and activates transcription when tetracycline or doxycycline is not present, whereas it cannot bind to the TRE promoter and activate the transcription when tetracycline or doxycycline is present. Thus, the expression of a TRAIL or a CD protein can be regulated by the addition or depletion of tetracycline or doxycycline.

The term “operably linked” means that a particular polynucleotide is linked to another polynucleotide so that it can conduct its function. The expression that a gene encoding a specific protein is operatively linked to a promoter implies that it is linked such that the gene can be transcribed into mRNA by the action of the promoter and translated into a protein.

When two or more genes are linked such that they are transcribed by one promoter in a lentiviral vector of the present invention, the vector may contain the ribosome entry site (IRES) so that each protein can be expressed from the single transcript.

The term “internal ribosome entry site (IRES)” refers to a nucleic acid sequence that functions to enable translation from an intermediate region of a transcript instead of the 5′-cap structure during eukaryotic protein synthesis. By using IRES, multiple proteins conducting different functions can be produced from a single transcript. According to one embodiment, the recombinant lentiviral vector of the present invention, in which a TRAIL protein and a CD protein are linked via IRES, can be transcribed into a single transcript, and then each protein can be produced therefrom.

The present invention provides a recombinant lentivirus comprising a gene encoding a TRAIL protein and a CD protein.

The recombinant lentivirus may be obtained by the steps of transforming a host cell with a lentiviral vector of the present invention, a packaging plasmid and an envelope plasmid; and isolating the lentivirus from the transformed host cell.

The terms “packaging plasmid” and “envelope plasmid” may provide helper constructs(e.g., plasmids or separated nucleic acid) for producing lentiviruses from the lentiviral vectors of the present invention for effective transfection. Such constructs contain useful elements for preparing and packaging lentiviral vectors in host cells. The above elements include a structural protein such as a gag precursor; a processing protein such as a pol precursor; protease; coat protein; and expression and regulatory signal necessary to prepare proteins and produce lentiviral particles in the host cell, etc.

For production of the recombinant lentivirus, Lenti-X Lentiviral Expression System provided by Clontech Laboratories Inc., a packaging plasmid (e.g., pRSV-Rev, psPAX, pCl-VSVG, pNHP, etc.) or an envelope plasmid (e.g., pMD2.G, pLTR-G, pHEF-VSVG, etc.) provided by Addgene can be used.

Further, the present invention provides a host cell transfected with the above recombinant lentivirus.

The term “transfection” refers to the delivery of a gene loaded in a recombinant lentiviral vector through viral infection.

A host cell according to the present invention may be a human embryonic stem cell (hES), a bone marrow stem cell (BMSC), a mesenchymal stem cell (MSC), a human neural stem cell (hNSCs), a limbal stem cell, or an oral mucosal epithelial cell. According to an embodiment of the present invention, the host cell may be a mesenchymal stem cell.

The term “mesenchymal stem cell (MSC)” refers to a multipotent stromal cell capable of differentiating into various cells including osteocytes, chondrocytes and adipocytes. Mesenchymal stem cells can differentiate into the cells of specific organs such as a bone, a cartilage, a fat, a tendon, a nervous tissue, fibroblasts and myocytes. These cells can be isolated or purified from adipose tissues, bone marrows, peripheral blood, umbilical cord blood, periosteum, dermis, mesodermal-derived tissues, and the like.

The host cell may be prepared by the following method:

1) primary infection of host cells with the lentiviruses comprising hTERT and c-myc genes;

2) secondary infection of the primary-infected host cells with the lentiviruses comprising a tTA gene; and

3) tertiary infection of the secondary-infected host cells with the lentiviruses comprising a TRAIL gene and a CD gene.

In the step 1), hTERT and c-myc are genes that immortalize host cells. Genes known as immortalizing genes other than hTERT and c-myc can also be used. According to one embodiment, the hTERT and c-myc proteins may be polypeptides having the amino acid sequences of SEQ ID NO: 9 and SEQ ID NO: 7, respectively. Meanwhile, the genes coding for the hTERT and c-myc proteins may be polynucleotides having the nucleotide sequences of SEQ ID NO: 10 and SEQ ID NO: 8, respectively.

In the step 2), tTA is a gene capable of regulating the expression of a target protein, which means tetracycline transactivator. The Tet-off system as used herein can regulate the expression of a target protein depending on the presence or absence of tetracycline or doxycycline as described above.

The tertiary infection of the step 2) can be carried out using a vector of the present invention which contains both TRAIL gene and CD gene in a single vector. However, in another aspect of the present invention, the TRAIL gene and CD genes can be prepared as each gene construct and inserted into two lentiviral vectors, respectively. That is, a lentiviral vector into which a gene construct prepared such that a gene encoding a TRAIL protein can be expressed is inserted, and a lentiviral vector into which a gene construct prepared such that a gene encoding a CD protein can be expressed is inserted can be used for the tertiary infection.

The prepared cells by the above method were designated as BM-03 and deposited on Jan. 6, 2017 under the Budapest Treaty on the International Recognition of the Deposit of Microorganisms for the Purposes of Patent Procedure at Korean Collection for Type Cultures (KCTC) at Korea Research Institute of Bioscience & Biotechnology (KRIBB) of 181, Ipsin-gil, Jeongeup-si, Jeollabuk-do, 56212, Republic of Korea and accorded with deposit number KCTC 13182BP on Jan. 17, 2017.

The present invention provides a pharmaceutical composition for preventing or treating cancer, comprising the above recombinant lentivirus or host cell as an active ingredient.

The cancer refers to a general cancer encompassing any blood cancer and solid cancer. Examples of cancer may include gastric cancer, colon cancer, breast cancer, lung cancer, non-small cell lung cancer, bone cancer, pancreatic cancer, skin cancer, head and neck cancer, melanoma, uterine cervix cancer, ovarian cancer, rectal cancer, endometrial cancer, Hodgkin's disease, a brain tumor, sarcoma, esophageal cancer, small bowel cancer, thyroid cancer, prostate cancer, leukemia, lymphoma, bladder cancer, a central nervous system tumor, a spinal cord tumor, etc.

The pharmaceutical composition is a kind of cell therapeutic agents, and may further comprise a pharmaceutically acceptable carrier. The carrier may be one generally used in the preparation of medicines, which may include lactose, dextrose, sucrose, sorbitol, mannitol, starches, gum acacia, calcium phosphate, alginates, gelatin, calcium silicate, microcrystalline cellulose, polyvinylpyrrolidone, cellulose, water, syrup, methylcellulose, methylhydroxybenzoate, propylhydroxybenzoate, talc, magnesium stearate, mineral oil, and the like.

In addition, the pharmaceutical compositions of the present invention may further comprise a pharmaceutically acceptable additive, which be selected from the group consisting of a lubricant, a wetting agent, a sweetening agent, a flavoring agent, an emulsifying agent, a suspending agent, a preservative and a combination thereof.

The carrier may be comprised in an amount of about 1% to about 99.99% by weight, preferably about 90% to about 99.99% by weight, based on the total weight of the pharmaceutical composition of the present invention, and the pharmaceutically acceptable additive may be comprised in an amount of about 0.1% to about 20% by weight.

The pharmaceutical composition may be prepared in a unit dosage form by formulating with a pharmaceutically acceptable carrier and excipient according to a conventional method, or may be prepared by filling into a multi-dose container. Herein, the formulation may be in the form of a solution, a suspension, a syrup or an emulsion in oil or aqueous media, or in the form of an extract, powders, a powdered drug, granules, tablets or capsules, and may additionally contain a dispersing or stabilizing agent.

The present invention also provides a method for preventing or treating cancer as described above, comprising the step of administering a pharmaceutical composition of the present invention to a subject.

The subject may be a mammal, particularly a human. The administration route and dosage of the pharmaceutical composition may be adjusted in various ways and amounts for administration to a subject depending on the condition of a patient and the presence of side effects, and the optimal administration method and dosage may be selected by a person skilled in the art in a suitable range. In addition, the pharmaceutical composition may be administered in combination with other drugs or physiologically active substances known to have a therapeutic effect on a disease to be treated, or may be formulated in a form of combination formulation with other drugs.

When the pharmaceutical composition is administered parenterally, examples of the administration include subcutaneous, ocular, intraperitoneal, intramuscular, oral, rectal, intraorbital, intracerebral, intracranial, intraspinal, intraventricular, intrathecal, intranasal, intravenous administration.

The above administration may be administered for one or more times, one to three times, specifically in two divided doses. In the case of repeated administrations, they can be administered at the interval of 12 to 48 hours, 24 to 36 hours, and more specifically, at the interval of 24 hours. In the case of lentiviruses, the administration may be conducted in an amount of 1.0×10⁶ to 1.0×10¹² TU, specifically 1.0×10⁸ to 1.0×10¹⁰ TU for adults. On the other hand, in the case of cells, the administration may be conducted in an amount of 1.0×10⁵ to 1.0×10¹¹ cells, specifically 1.0×10⁷ to 1.0×10⁹ cells for adults. When the dose is high, the administration may be conducted several times a day.

### Mode for the Invention

Hereinafter, the present invention is described in detail with reference to the following examples. However, the following examples are intended to illustrate the present invention, and the present invention is not limited thereto.

### EXAMPLE 1

### Preparation of Immortalized Mesenchymal Stem Cells (MSCs)

1-1. Preparation of Lentiviral Vectors Ccontaining Immortalized Gene

In order to immortalize MSCs, lentiviral vectors respectively containing c-Myc and hTERT, which are immortalized genes, were prepared. Herein, a gene construct expressing the tTA protein was inserted together to use the Tet-off system.

First, a pBD lentiviral vector was prepared by substituting the EF promoter in the expression cassette of the pWPT vector (Addgene, USA) with the CMV promoter, and adding the RSV promoter to the downstream thereof.

The c-Myc gene (SEQ ID NO: 8) and thymidine kinase (TK) gene (SEQ ID NO: 6) were linked via IRES and inserted into the pBD lentiviral vector so that the expression can be regulated by the CMV promoter. The constructed vector was designated as pBD-1.

On the other hand, the hTERT gene (SEQ ID NO: 10) was inserted into the pBD lentiviral vector such that the expression can be regulated by the CMV promoter. A gene having resistance to zeomycin (ZeoR; SEQ ID NO: 16) was inserted thereto such that the expression can be regulated by the RSV promoter. The constructed vector was designated as pBD-2.

In addition, a tTA (tetracycline transactivator) gene (SEQ ID NO: 12) was inserted into the pBD lentiviral vector such that the expression can be regulated by the CMV promoter. A gene having resistance to puromycin (PuroR; SEQ ID NO: 14) was inserted thereto such that the expression can be regulated by the RSV promoter. The constructed vector was designated as pBD-3.

1-2. Production of Lentiviruses Containing Immortalized Gene

Using the lentiviral vectors constructed in Example 1-1, lentiviruses containing immortalized genes were produced by the following method.

First, Lenti-X cells (Clontech Laboratories, USA) were cultured in a 150 mm dish using DMEM supplemented with 10% fetal bovine serum. Meanwhile, lentiviral vectors were extracted and quantified from DH5a E. coli cells using EndoFreePlasmin Plasmid Maxi Kit (Qiagen, USA).

The cultured Lenti-X cells were washed with PBS, and then 3 ml of TrypLE™ Select CTS™ (Gibco, USA) was added thereto. The cells were left at 37° C. for about 5 minutes, and then their detachment was verified. The detached cells were neutralized by adding 7 ml of DMEM supplemented with 10% fetal bovine serum thereto. The neutralized cells were collected in a 50 ml tube and centrifuged at 1,500 rpm for 5 minutes. The resultant supernatant was removed and the cells were resuspended by adding 10 ml of DMEM supplemented with 10% fetal bovine serum thereto. The suspended cells were counted with a hematocytometer and then dispensed into a 150 mm dish in an amount of 1.2×10⁷ cells. When the dispensed cells were cultured to a cell saturation of about 90%, the cells were transformed with a mixture of 12 μg of lentiviral vectors, 12 μg of psPAX (Addgene; expressing gag-pol, packaging plasmid) and 2.4 μg of pMD.G plasmid (Addgene; expressing VSVG, envelope plasmid). In order to facilitate the transformation, lipofectamine (Invitrogen, USA) and PLUS reagent (Invitrogen, USA) were used. 6 hours after the transformation, the medium was replaced with DMEM supplemented with 10% fetal bovine serum. After 48 hours of additional culturing, the supernatant was collected.

The obtained supernatant was mixed with a lentivirus concentration kit (Lenti-X concentrator, Clontech Laboratories, USA) and then cultured overnight at 4° C. It was centrifuged under the condition of 4° C. and 4,000 rpm for 2 hours to obtain viruses, which were then resuspended in 0.5 ml of DMEM not containing FBS. As a result, lentiviruses produced from pBD-1, pBD-2 and pBD-3 lentiviral vectors were prepared at the concentrations of 4.0×10⁸ TU/ml, 2.0×10⁸ TU/ml and 1.2×10⁹ TU/ml, respectively.

1-3. Preparation of Immortalized Mesenchymal Stem Cells

Immortalized MSCs were prepared using the lentiviruses containing the immortalized genes produced in Example 1-2.

First, bone marrow-derived MSCs were prepared by the following method. Specifically, bone marrow aspirate was obtained from the iliac crest of a healthy donor. The aspirate was mixed with 20 IU/ml heparin in a sterile container to inhibit coagulation. The bone marrow mixture solution was centrifuged under the condition of 4° C., 739 g for 7 minutes, and then the supernatant was removed and the resultant was mixed with 10-fold amount (in volume) of sterilized water. The resultant mixture was centrifuged again under the same condition to obtain cell pellets. The obtained pellets were suspended in DMEM-low glucose (11885-084, Gibco, USA) supplemented with 20% FBS and 5 ng/ml b-FGF (100-18B, Peprotech, USA), which were then dispensed into a culture flask. It was cultured under the condition of 37° C., 5% CO₂ for 24 to 48 hours, and then replaced with a new medium. The cells were cultured with passages while the medium was replaced with new one at the interval of 3 to 4 days. After 2 weeks of culturing, MSCs were confirmed using a fluorescent cell analyzer.

The MSCs prepared above were infected with the pBD-1 lentiviruses produced in Example 1-2 at 100 MOI using Retronectin (Clontech Laboratories, USA). The infected cells were infected with the pBD-2 lentiviral vector at 100 MOI by the same method. After infection, the cells infected with pBD-2 lentiviruses were selected by adding 500 μg/ml zeomycin to the culture medium of the stabilized cells.

The selected cells were infected with pBD-3 lentiviral vector at 100 MOI. After infection, the cells infected with pBD-3 lentiviruses were selected by adding 1 μg/ml puromycin to the culture medium of the stabilized cells.

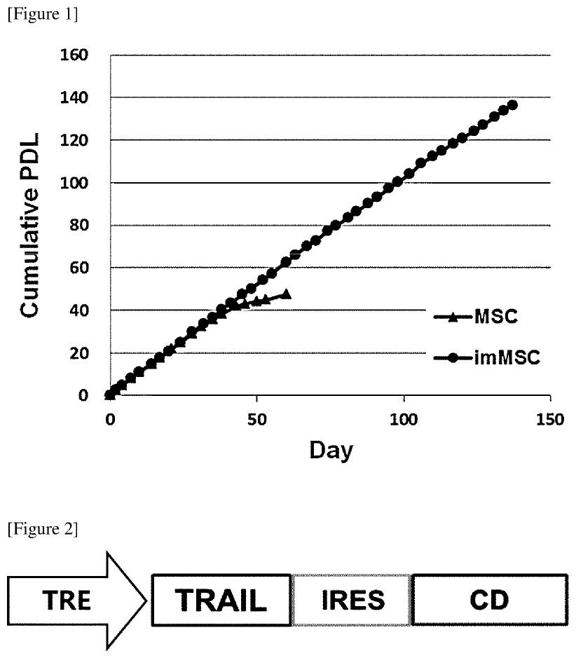

As a result, the cell proliferation rates of the MSCs containing the immortalized gene and the MSCs not containing the immortalized gene are shown in FIG. 1. As shown in FIG. 1, the MSCs infected with lentiviruses containing the immortalized genes, c-myc and hTERT, maintained high cell proliferation rates even after 120 days of culture. On the other hand, the cell proliferation rate of non-immortalized MSCs(MSC) decreased rapidly after 40 days of culture.

### EXAMPLE 2

### Construction of Lentivirus Containing TRAIL and CD Genes

2-1. Construction of Lentiviral Vector Containing TRAIL and CD Genes

The TRAIL gene (SEQ ID NO: 2) and the CD gene (SEQ ID NO: 4) were inserted into the prepared pBD lentiviral vector. Herein, the inserted TRAIL and CD genes were linked via IRES (internal ribosome entry site), and the expression was designed to be regulated by the TRE promoter. IRES is a ribosome binding site that allows translation to begin without the 5′-cap structure, enabling the expression of two proteins by a single mRNA. On the other hand, the TRE promoter can regulate the expression of the gene linked to the promoter depending on the presence or absence of the addition of doxycycline.

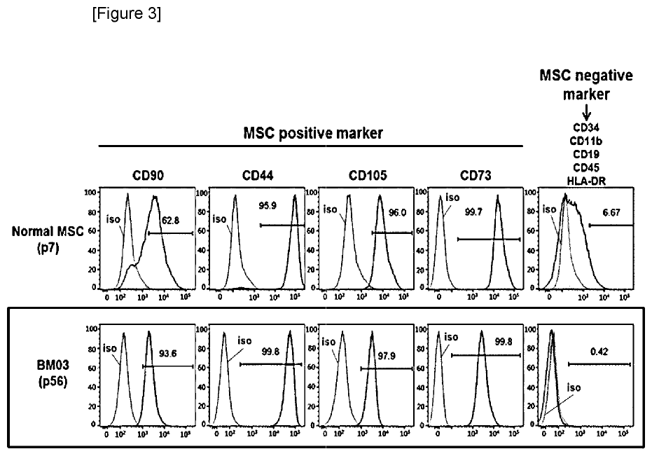

The constructed vector was designated as pBD-4, and the structure of the gene construct is shown in FIG. 2.

2-2. Production of Lentivirus Containing TRAIL and CD Genes

Using the lentiviral vector containing the TRAIL and CD genes constructed in Example 2-1, lentivirus was produced by the same method as described in Example 1-2. The lentivirus produced was prepared at a concentration of 7.6×10⁸ TU/ml.

### EXAMPLE 3

### Preparation of MSC Infected with Lentivirus Containing TRAIL and CD Genes

3-1. Preparation of MSC Infected with Lentivirus Containing TRAIL and CD Genes

Cells expressing TRAIL and CD genes were prepared by infecting the immortalized MSCs prepared in Example 1-3 with lentivirus containing TRAIL and CD genes produced in Example 2-2. The infection was carried out by the same method as described in Examples 1-3. After the infection, 1 μg/ml of doxycycline (631311, Clontech, USA) was added to the culture medium of the stabilized cells, and the cells were cultured in a state in which the expressions of TRAIL and CD were suppressed. After the cells were stabilized, TRAIL and CD expressions were induced by culturing the cells in cell media for 72 hours from which doxycycline was removed. Then the cells expressing TRAIL on the surface are isolated using FACS.

Specifically, the cells infected with lentivirus containing TRAIL and CD genes were dispensed into FACS tubes at 5×10⁵ cells/tube, and centrifuged under the condition of 4° C. and 1,500 rpm for 5 minutes, and the resulting supernatant was removed. The cells were resuspended by adding 1 ml of FACS buffer (PBS containing 2% fetal bovine serum) thereto, and centrifuged under the same condition and the resulting supernatant was removed. After the above washing procedure was conducted once more, the cells were resuspended in 1 ml of FACS buffer. The resuspended cells were added with a mixture prepared by adding 0.3 μl of LIVE/DEAD® Fixable Near-IR Dead Cell Stain (Life Technologies-Molecular Probes, USA) and and 5μl of APC anti-human CD253 antibody (BioLegend, Cat #. 308210, USA), an anti-TRAIL antibody, to 200 μl of FACS buffer, and the resultant mixture was reacted at 4° C. for 30 minutes. After the reaction, the cells were washed twice by the same method as above and the resulting supernatant was removed. 300 μl of fixing buffer (PBS containing 2% formaldehyde and 1% fetal bovine serum) was added to the washed cells and left at 4° C. for at least 15 minutes. The cells were analyzed by the FACS (LSRFortessa, BD biosciences, USA) device and cells expressing TRAIL were isolated.

The isolated cells were cultured to form colonies. The monoclonal cells obtained from the colonies formed were cultured to establish a cell line, which was designated as BM-03. The cell line BM-03 was deposited on Jan. 6, 2017 under the Budapest Treaty on the International Recognition of the Deposit of Microorganisms for the Purposes of Patent Procedure at Korean Collection for Type Cultures (KCTC) at Korea Research Institute of Bioscience & Biotechnology (KRIBB) of 181, Ipsin-gil, Jeongeup-si, Jeollabuk-do, 56212, Republic of Korea and accorded with deposit number KCTC 13182BP on Jan. 17, 2017.

3-2. Examination of Surface Antigen Protein Expression in Established Cell Lines

Surface antigen protein expression of bone marrow-derived MSCs before gene insertion and that of BM-03 cell line to which TRAIL and CD genes were inserted were analyzed using a human MSC assay kit (Stemflow™, Cat No 562245, BD). Experiments were conducted according to the manual included in each kit, and the experimental results are shown in FIG. 3.

As shown in FIG. 3, it was verified that the expressions of surface antigen proteins CD90, CD44, CD105, and CD73, inherent characteristics of MSCs, in BM-03 cell line which went through the gene manipulation to express TRAIL and CD therapeutic genes were the same as those in bone marrow-derived MSCs.

3-3. Examination of Differentiation Ability of BM-03 Cell Line

BM-03 cell line was seeded in a 12-well plate at a concentration of 1×10⁴ cells/cm² to examine adipogenesis. Using general culture medium, the cells were cultured in an incubator under the condition of 5% CO₂ and 37° C. for 2 to 3 days, and then replaced with a adipogenesis differentiation medium (StemPro® adipogenesis differentiation kit, Thermo fisher Scientific, A10070-01). The cells were cultured for 21 days while the medium was changed every 3 or 4 days. When the culture was completed, the cells were stained with Oil Red 0 solution and examined with a microscope.

To examine osteogenesis, cells were seeded in a 12-well plate at a concentration of 5×10³ cells/cm². Using general culture medium, the cells were cultured in an incubator under the condition of 5% CO₂ and 37° C. for 2 to 3 days, and then replaced with an osteogenesis differentiation medium (StemiPro® osteogenesis differentiation kit, Thermo fisher Scientific, A10072-01). The cells were cultured for 21 days while the medium was changed every 3 or 4 days. When the culture was completed, the cells were stained with Alizarin Red S and examined with a microscope.

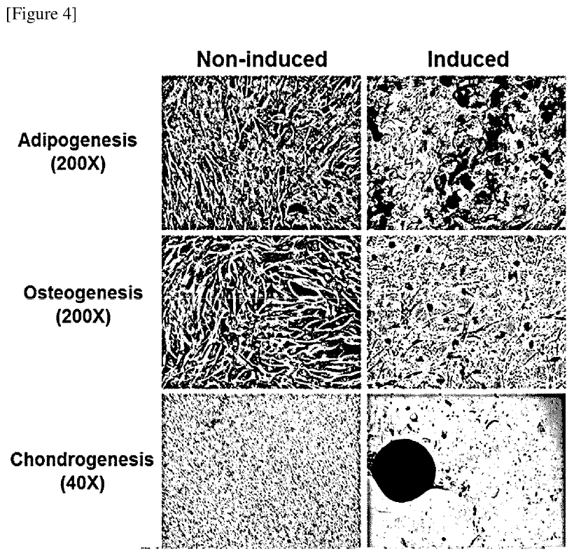

To examine chondrogenesis, cells were suspended at a concentration of 1.6×10⁷ cells/ml, and 5 μl of the suspension was seeded in a 24-well plate and cultured for 2 hours. 500 μl of chondrogenesis differentiation medium (StemiPro® chondrogenesis differentiation kit, Thermo fisher Scientific, A10071-01) was added thereto, and the cells were cultured for 14 days while the medium was changed every 3 days. After the culture, the medium was aspirated and the cells were rinsed with DPBS to take out pellets. After cryosection, the cells were stained with Alcian blue and examined by a microscope. The results are shown in FIG. 4.

As shown in FIG. 4, it was verified that BM-03 cell line which went through the gene manipulation to express TRAIL and CD therapeutic genes had differentiation ability which is an inherent characteristic of MSCs.

3-4. Test for Examination of Gene Introduced into BM-03 Cell Line

A sample of the established cell line, BM-03, was thawed for about 1 minute in a constant temperature water bath at 37° C., transferred to a 15 ml tube containing 9 ml PBS, and subjected to a Cell Down process for 5 minutes at 1,500 rpm. After PBS was completely removed, the pellet was suspended in 200 μl of PBS in a 1.5 ml tube, and transferred. gDNA was prepared using NucleoSpin® Tissue (MN, 740952.250), and the mixture was prepared as shown in Table 1 below, followed by PCR by the steps shown in Table 2 below. Herein, 100 ng of BM-03 plasmid DNA was added as a positive control and 1 μl of dW was used as a negative control.

TABLE 1

| | |
|---|---|
| Forward primer (SEQ ID NO: 17) | 1 μl |
| (10 pmol/μl, GX535) | |
| Reverse primer (SEQ ID NO: 18) | 1 μl |
| (10 pmol/μl, GX534) | |
| Sample (100 ng/μl) | 1 μl |
| dW | 17 μl |
| Total volume | 20 μl |

TABLE 2

| Step | Temperature | Time | Cycle |
|---|---|---|---|
| 1st | 95° C. | 5 min | 1 |
| 2nd | 95° C. | 45 sec | 35 |
| | 58° C. | 45 sec | |
| | 72° C. | 1 min | |
| 3rd | 72° C. | 7 min | 1 |
| 4th | 4° C. | Indefinitely | 1 |

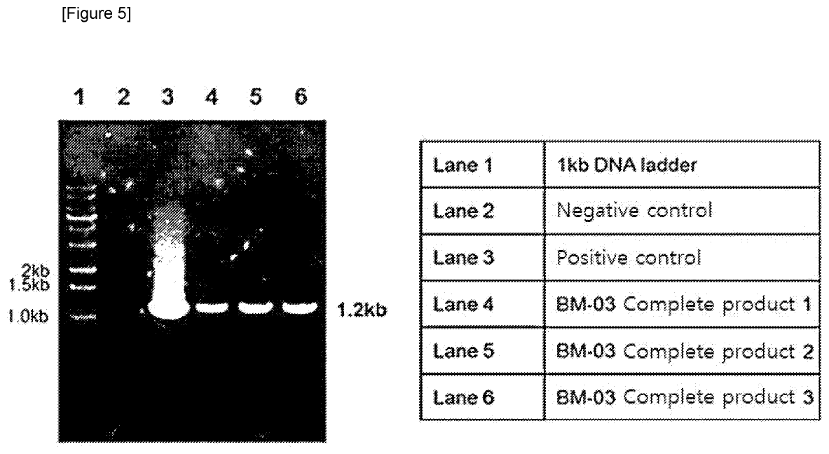

1% agarose gel was placed in an electrophoresis kit. 10 μl of DNA Size Marker was loaded in the first well and 10 μl each of a negative control, a positive control, and BM-03 sample (×3) were loaded in the following wells respectively in the above order. Thereafter, electrophoresis was conducted at 100 V for 20 minutes, and a gel photograph was taken. The result is shown in FIG. 5.

As shown in FIG. 5, all three of the BM-03 cell line samples showed PCR products of the same size (1.2 kb) as the positive control.

3-5. Identification of Expression of TRAIL and CD Proteins in Established Cell Lines

In the BM-03 cell line established in Example 3-1, the expression of TRAIL and CD proteins were induced by the same method as in Example 3-1, and FACS was conducted to identify the expression of TRAIL according to the presence or absence of doxycycline. Then, the expression of CD protein was identified in the cell line in which the expression of TRAIL was identified.

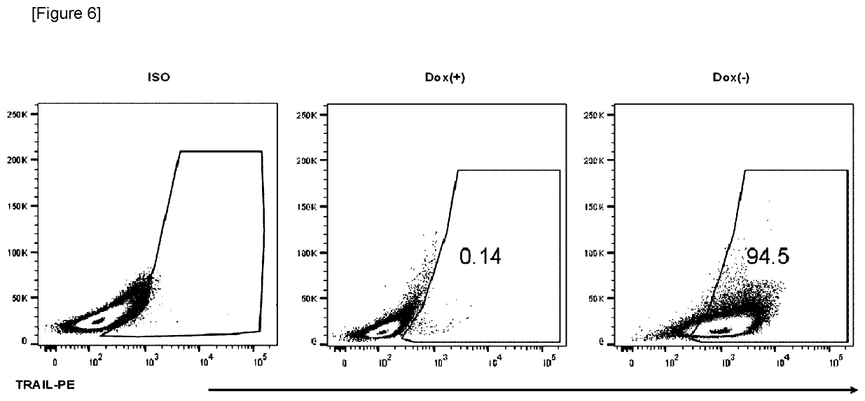

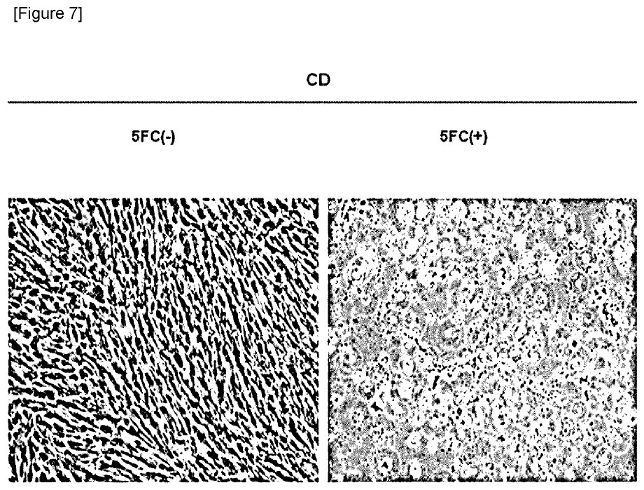

Specifically, the BM-03 cell line was seeded in T75 flasks at a number of 5×10⁵ cells using DMEM supplemented with 10% FBS containing 2 μg/ml of doxycycline, and DMEM supplemented with 10% FBS not containing 2 μg/ml of doxycycline. Cells were recovered after 3 days of culture. After measuring the number of cells, 5×10⁵ cells per group were stained with PE anti-human CD253 (TRAIL) antibody (BioLegend, 308206) and PE Mouse IgG1, κIsotype Control Antibody (BioLegend, 400112). In addition, to identify the expression after excluding dead cells, LIVE/DEAD® Fixable Near-IR Dead Cell Stain Kit (Thermos Fisher Scientific L34976) antibody was used. Samples were analyzed using FACS (LSRFortessa, BD Biosciences) device. After the cells were cultured by the same method, the expression of CD was induced by removing doxycycline. 48 hours after adding 5-FC (5-fluorocytosine, Sigma) thereto at a concentration of 100 μg/ml, apoptosis was observed. The results are shown in FIG. 6 and FIG. 7.

As shown in FIG. 6, it was found that TRAIL was not expressed when doxycycline was added, but TRAIL was expressed in the BM-03 cell line cultured in the medium from which the doxycycline was removed. Further, it was found that, as shown in FIG. 7, if the CD protein was expressed, cells were killed by SFC. That is, the cells die owing to the expression of the CD protein. Accordingly, it was found that the TRAIL protein and CD protein were expressed in the prepared mesenchymal stem cells, which was regulated by the Tet-off system.

3-6. Cell Population Doubling Level (PDL) Analysis

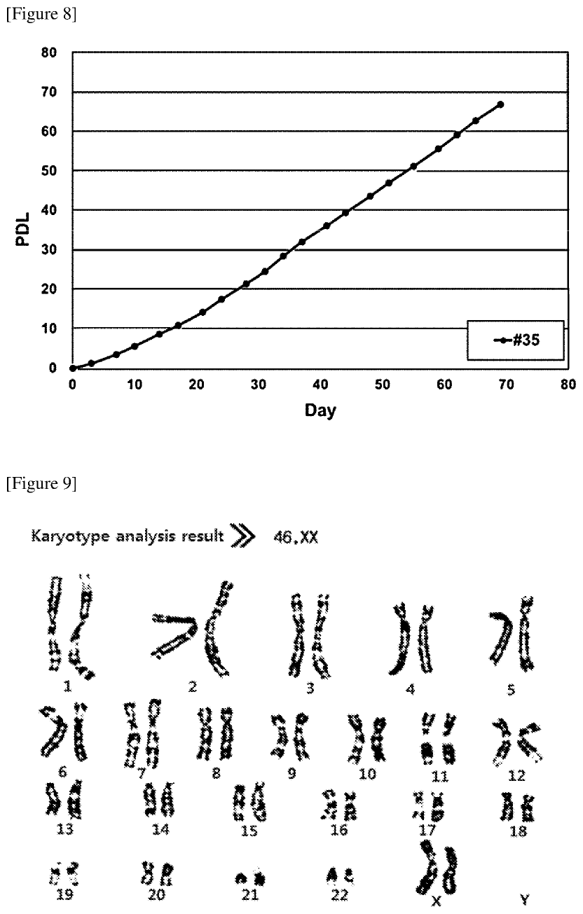

The BM-03 cell line was seeded in a T75 flask at 4×10⁵ cells using a medium containing 2 μg/ml of doxycycline. Cells were obtained through subculture for about 3 or 4 days, and the total number of cells was counted. Cells of the same number were seeded, and PDL was measured every 3 or 4 days. The PDL was calculated using the following Equation 1, and the result is shown in FIG. 8. In the Equation 1, X represents the initial PDL, I represents the initial number of cells seeded in the blood vesselmedium, Y represents the final cell yield or the number of cells in the end of growth period.

PDL=X+3.222 (log Y−log I) [Equation 1]

As shown in FIG. 8, stable growth was observed in long-term subculture.

3-7. Karyotype Analysis of Cells

In order to examine the chromosomal abnormality of the cells to which genes were introduced into the BM-03 cell line, analysis was requested to EONS Life Science Institute (Korea) and conducted in accordance with the protocol. The result of the analysis is shown in FIG. 9.

As shown in FIG. 9, no abnormality was observed in the chromosome of the BM-03 cells into which genes were introduced, and thus it was determined as a normal karyotype.

1. A recombinant lentiviral vector comprising a gene encoding a TNF-related apoptosis-inducing ligand (TRAIL) protein, and a cytosine deaminase (CD) protein.

2. The recombinant lentiviral vector according to claim 1, wherein the TRAIL protein is a polypeptide having the amino acid sequence of SEQ ID NO: 1.

3. The recombinant lentiviral vector according to claim 1, wherein the CD protein is a polypeptide having the amino acid sequence of SEQ ID NO: 3.

4. The recombinant lentiviral vector according to claim 1, which comprises one or two promoters.

5. The recombinant lentiviral vector according to claim 4, wherein the promoter is a cytomegalovirus (CMV), respiratory syncytial virus (RSV), human elongation factor-1 alpha (EF-1a) or tetracycline response elements (TRE) promoter .

6. The recombinant lentiviral vector according to claim 1, which comprises an internal ribosome entry site (IRES).

7. A recombinant lentivirus comprising a gene encoding a TRAIL protein and a CD protein.

8. The recombinant lentivirus according to claim 7, wherein the lentivirus is obtained by a process comprising steps of:
transforming a host cell with a lentiviral vector, a packaging plasmid, and an envelope plasmid; and
isolating the lentivirus from the transformed host cell,
wherein the recombinant lentiviral vector comprises a gene encoding a TNF-related apoptosis-inducing ligand (TRAIL) protein, and a cytosine deaminase (CD) protein.

9. A host cell transfected with the recombinant lentivirus according to claim 7.

10. The host cell according to claim 9, which is a mesenchymal stem cell.

11. A pharmaceutical composition comprising the recombinant lentivirus according to claim 7 as an active ingredient.

12. A pharmaceutical composition comprising the host cell according to claim 9 as an active ingredient.

13. A method for preventing or treating a cancer of a subject in need thereof comprising administering a recombinant lentivirus or a host cell transfected with the recombinant lentivirus, as an active ingredient, to the subject, wherein the recombinant lentivirus comprises the recombinant lentiviral vector of claim 1 encoding a TNF-related apoptosis-inducing ligand (TRAIL) protein, and a cytosine deaminase (CD) protein.

14. The method according to claim 13, wherein the vascular disease is selected from the group consisting of angina pectoris, myocardial infarction, arteriosclerosis, atherosclerosis, periarteritis nodosa, Takayasu's arteritis, vascular occlusion, stroke, cerebral hemorrhage, cerebral infarction, cerebral edema, and ischemic diseases.

15. The method of claim 13, wherein the host cell is mesenchymal stem cell.

16. The method of claim 13, wherein the TRAIL protein is a polypeptide having the amino acid sequence of SEQ ID NO: 1.

17. The method of claim 13, wherein the CD protein is a polypeptide having the amino acid sequence of SEQ ID NO: 3.

18. The method of claim 13, wherein the recombinant lentiviral vector comprises a plasmid selected from the group consisting of a cytomegalovirus (CMV), respiratory syncytial virus (RSV), human elongation factor-1 alpha (EF-1 α) or tetracycline response elements (TRE) promoter.


SEQUENCE LISTING (18 sequences)

SEQ ID NO: 1 - protein, 281 aa, Artificial Sequence [sequence omitted]

SEQ ID NO: 2 - DNA, 846 bp, Artificial Sequence
  nucleotide sequence of TRAIL
atggcaatgatggaggtccagggggggccttcactgggacagacatgcgtgctgatcgtcatctttaccgtgctgctgcagtcactgtgcgtggctgtcacatacgtgtatttcactaacgagctgaagcagatgcaggacaagtactcaaaaagcggaatcgcatgctttctgaaagaagacgatagctattgggaccctaacgatgaggaatccatgaactccccatgttggcaggtgaagtggcagctgcgacagctggtccggaaaatgatcctgaggactagtgaggaaactatttcaaccgtgcaggagaagcagcagaatatcagcccactggtgcgggaaagaggaccacagcgagtcgcagctcacattaccggaacaaggggccgcagcaacaccctgagctccccaaactccaagaatgagaaagccctgggcagaaagatcaattcctgggaatctagtaggagtgggcactcattcctgagcaacctgcatctgcgcaatggggagctggtcatccatgaaaaaggcttctactatatctactctcagacctatttccgatttcaggaggaaattaaggagaacacaaagaatgacaaacagatggtccagtacatctataaatacacatcttaccccgatcctattctgctgatgaagagtgcacggaactcctgttggtctaaagacgccgagtatgggctgtacagcatctatcagggcgggattttcgagctgaaggaaaatgatagaatctttgtgagcgtcaccaacgagcatctgattgacatggatcacgaggcttcatttttcggggcattcctggtcggataa

SEQ ID NO: 3 - protein, 373 aa, Artificial Sequence
  amino acid sequence of CD::UPRT
MVTGGMASKWDQKGMDIAYEEAALGYKEGGVPIGGCLINNKDGSVLGRGHNMRFQKGSATLHGEISTLENCGRLEGKVYKDTTLYTTLSPCDMCTGAIIMYGIPRCVVGENVNFKSKGEKYLQTRGHEVVVVDDERCKKIMKQFIDERPQDWFEDIGESSEPFKNVYLLPQTNQLLGLYTIIRNKNTTRPDFIFYSDRIIRLLVEEGLNHLPVQKQIVETDTNENFEGVSFMGKICGVSIVRAGESMEQGLRDCCRSVRIGKILIQRDEETALPKLFYEKLPEDISERYVFLLDPMLATGGSAIMATEVLIKRGVKPERIYFLNLICSKEGIEKYHAAFPEVRIVTGALDRGLDENKYLVPGLGDFGDRYYCV

SEQ ID NO: 4 - DNA, 1122 bp, Artificial Sequence
  nucleotide sequence of CD::UPRT
atggtgaccggcggcatggccagcaagtgggaccagaagggcatggacatcgcctacgaggaggccgctctgggctacaaggagggcggcgtgcccatcggcggatgcctgatcaacaacaaggacggcagcgtgctgggcagaggccacaacatgagattccagaagggcagcgccaccctgcacggcgagatcagcaccctggagaactgcggcagactggagggcaaggtgtacaaggacacaaccctgtacaccacactgagcccctgcgacatgtgcaccggcgccatcatcatgtacggcatccccagatgcgtggtgggcgagaacgtgaacttcaagagcaagggcgagaagtacctgcagaccagaggccacgaggtggtcgtcgtggacgacgagagatgcaagaagatcatgaagcagttcatcgacgagagaccccaggactggttcgaggacatcggcgagagcagcgagcccttcaagaacgtgtacctgctgccccagaccaaccagctgctgggcctgtacaccatcatcagaaacaagaacaccaccagacccgacttcatcttctacagcgacagaatcatcagactgctggtggaggagggcctgaaccacctgcccgtgcagaagcagatcgtggagaccgacaccaacgagaacttcgagggcgtgagcttcatgggcaaaatctgcggcgtgagcatcgtgagagccggcgagagcatggagcagggcctgagagactgctgcagaagcgtgagaatcggcaagatcctgatccagagagacgaggagaccgccctgcccaagctgttctacgagaagctgcccgaggacatcagcgagagatacgtgttcctgctggaccccatgctggccaccggcggcagcgccatcatggccaccgaggtgctgatcaagagaggcgtgaagcccgagagaatctacttcctgaacctgatctgcagcaaggagggcatcgagaagtaccacgctgccttccccgaggtgagaatcgtgaccggcgccctggacagaggcctggacgagaacaagtacctggtgcccggcctgggcgacttcggcgacagatactactgcgtgtaa

SEQ ID NO: 5 - protein, 376 aa, Artificial Sequence
  amino acid sequence of TK
MASYPCHQHASAFDQAARSRGHSNRRTALRPRRQQEATEVRLEQKMPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHVGGEAGSSHAPPPALTLIFDRHPIAALLCYPAARYLMGSMTPQAVLAFVALIPPTLPGTNIVLGALPEDRHIDRLAKRQRPGERLDLAMLAAIRRVYGLLANTVRYLQGGGSWWEDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEAN

SEQ ID NO: 6 - DNA, 1131 bp, Artificial Sequence
  nucleotide sequence of TK
atggccagctacccctgtcaccagcacgccagcgccttcgaccaggccgctagaagcagaggccacagcaacagaagaaccgccctgagacccagaagacagcaggaggccacagaggtgagactggagcagaagatgcccaccctgctgagagtgtacatcgatggaccccacggcatgggcaagaccacaacaacccagctgctggtggccctgggcagcagagacgacatcgtgtacgtgcccgagcccatgacctactggcaggtgctgggagccagcgagaccatcgccaacatctacaccacacagcacagactggaccagggcgagatcagcgccggcgacgctgccgtggtgatgaccagcgcccagatcacaatgggcatgccctacgccgtgaccgatgccgtgctggctccccacgtgggcggagaggccggcagcagccacgcccctccccctgccctgaccctgatcttcgacagacaccccatcgccgccctgctgtgctaccccgccgctagatacctgatgggcagcatgacaccccaggccgtgctggccttcgtggccctgatcccccctaccctgcccggcaccaacatcgtgctgggcgccctgcccgaggacagacacatcgacagactggctaagagacagagacccggcgagagactggacctggccatgctggccgccatcagaagagtgtacggcctgctggccaacaccgtgagatacctgcagggaggcggcagctggtgggaggactggggccagctgagcggcaccgccgtgcctccccagggcgccgagccccagagcaacgccggccctagaccccacatcggcgacaccctgttcaccctgtttagagcccccgagctgctggcccccaacggcgacctgtacaacgtgttcgcctgggccctggacgtgctggccaagagactgagacccatgcacgtgttcatcctggactacgaccagagccccgccggctgcagagatgccctgctgcagctgaccagcggcatggtgcagacccacgtgaccacacccggcagcatccccaccatctgcgacctggccagaacctttgccagagagatgggcgaggccaactga

SEQ ID NO: 7 - protein, 454 aa, Artificial Sequence
  amino acid sequence of c-myc
MDFFRVVENQQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 8 - DNA, 1365 bp, Artificial Sequence
  nucleotide sequence of c-myc
atggatttctttcgcgtcgtcgagaaccagcagccacccgccactatgcctctgaacgtgtcttttactaacaggaactatgatctggattacgacagcgtgcagccctacttctattgcgatgaggaagagaacttttatcagcagcagcagcagagcgagctgcagccacctgcaccttccgaagacatttggaagaaattcgagctgctgcctacaccacccctgtctccaagtcggagaagcggcctgtgttcacccagctacgtggccgtcactcctttcagcctgaggggggacaatgatggcgggggaggctccttttctacagccgatcagctggaaatggtgactgagctgctggggggagacatggtcaaccagagcttcatttgcgatcctgacgatgaaacttttatcaagaacatcatcatccaggactgtatgtggtcaggctttagcgccgctgcaaagctggtgtctgagaaactggcaagttatcaggccgctcggaaagatagtgggtcacctaacccagctagaggacactccgtgtgctctacaagctccctgtacctgcaggacctgagcgcagccgcttccgagtgtattgatccctccgtggtcttcccctatcctctgaatgactctagttcacccaagagttgcgcatcacaggacagctccgccttttcaccttctagtgatagcctgctgtcaagcactgaatcctctccacagggcagcccagagccactggtgctgcatgaagagacccctccaaccacaagttcagattccgaagaggaacaggaggacgaggaagagatcgatgtggtctctgtggaaaagcgccaggctccaggaaaacgaagcgagtccggctctccaagtgcaggaggacactccaagccacctcattctcccctggtgctgaaaaggtgccacgtctccacccaccagcataactacgcagccccaccctctacacgaaaggactatccagctgcaaagcgcgtgaaactggatagcgtgagagtcctgaggcagatcagtaacaatcggaagtgtacttcacccagaagctccgacaccgaagagaacgtgaaaaggcgcacccataatgtcctggaacgccagcgacggaatgagctgaagaggtccttctttgccctgcgcgatcagattcctgaactggagaacaatgagaaggctccaaaagtggtcattctgaagaaagccacagcttacatcctgtctgtgcaggccgaagagcagaaactgatcagtgaagaggacctgctgagaaaacgcagggaacagctgaaacataaactggaacagctgagaaactcttgtgcttaa

SEQ ID NO: 9 - protein, 1132 aa, Artificial Sequence
  amino acid sequence of TERT
MPRAPRCRAVRSLLRSHYREVLPLATFVRRLGPQGWRLVQRGDPAAFRALVAQCLVCVPWDARPPPAAPSFRQVSCLKELVARVLQRLCERGAKNVLAFGFALLDGARGGPPEAFTTSVRSYLPNTVTDALRGSGAWGLLLRRVGDDVLVHLLARCALFVLVAPSCAYQVCGPPLYQLGAATQARPPPHASGPRRRLGCERAWNHSVREAGVPLGLPAPGARRRGGSASRSLPLPKRPRRGAAPEPERTPVGQGSWAHPGRTRGPSDRGFCVVSPARPAEEATSLEGALSGTRHSHPSVGRQHHAGPPSTSRPPRPWDTPCPPVYAETKHFLYSSGDKEQLRPSFLLSSLRPSLTGARRLVETIFLGSRPWMPGTPRRLPRLPQRYWQMRPLFLELLGNHAQCPYGVLLKTHCPLRAAVTPAAGVCAREKPQGSVAAPEEEDTDPRRLVQLLRQHSSPWQVYGFVRACLRRLVPPGLWGSRHNERRFLRNTKKFISLGKHAKLSLQELTWKMSVRDCAWLRRSPGVGCVPAAEHRLREEILAKFLHWLMSVYVVELLRSFFYVTETTFQKNRLFFYRKSVWSKLQSIGIRQHLKRVQLRELSEAEVRQHREARPALLTSRLRFIPKPDGLRPIVNMDYVVGARTFRREKRAERLTSRVKALFSVLNYERARRPGLLGASVLGLDDIHRAWRTFVLRVRAQDPPPELYFVKVDVTGAYDTIPQDRLTEVIASIIKPQNTYCVRRYAVVQKAAHGHVRKAFKSHVSTLTDLQPYMRQFVAHLQETSPLRDAVVIEQSSSLNEASSGLFDVFLRFMCHHAVRIRGKSYVQCQGIPQGSILSTLLCSLCYGDMENKLFAGIRRDGLLLRLVDDFLLVTPHLTHAKTFLRTLVRGVPEYGCVVNLRKTVVNFPVEDEALGGTAFVQMPAHGLFPWCGLLLDTRTLEVQSDYSSYARTSIRASLTFNRGFKAGRNMRRKLFGVLRLKCHSLFLDLQVNSLQTVCTNIYKILLLQAYRFHACVLQLPFHQQVWKNPTFFLRVISDTASLCYSILKAKNAGMSLGAKGAAGPLPSEAVQWLCHQAFLLKLTRHRVTYVPLLGSLRTAQTQLSRKLPGTTLTALEAAANPALPSDFKTILD

SEQ ID NO: 10 - DNA, 3399 bp, Artificial Sequence
  nucleotide sequence of hTERT
atgcccagagctcccagatgcagagccgtgagaagcctgctgagaagccactacagagaggtgctgcccctggccaccttcgtgagaagactgggaccccagggctggagactggtgcagagaggcgaccccgcagcctttagagccctggtggcccagtgcctggtgtgcgtgccctgggacgccagacctcctcccgctgcccccagcttcagacaggtgagctgcctgaaggagctggtggccagagtgctccagagactgtgcgagagaggcgccaagaacgtgctggcctttggcttcgccctgctggatggagccagaggcggacctcccgaggccttcaccacaagcgtgagaagctacctgcccaacaccgtgaccgatgccctgagaggctccggcgcctggggcctgctcctgagaagagtgggcgacgacgtgctggtgcacctgctggccagatgcgccctgttcgtgctggtggctcccagctgcgcctaccaggtgtgcggaccccctctgtaccagctgggagccgccacccaggcaagaccccctccccacgcctctggacccagaagaagactgggctgcgagagagcctggaaccacagcgtgagagaggctggcgtgcccctgggcctgcccgcccctggcgccagaagaagaggcggcagcgccagcagaagcctgcccctgcccaagagacccagacgcggcgccgctcccgagcctgagagaacacccgtgggccagggcagctgggcccaccccggcagaaccagaggacccagcgacagaggcttctgcgtggtgagccctgccagacccgccgaggaggccaccagcctggagggcgccctgagcggcaccagacacagccaccccagcgtgggcagacagcaccacgccggccctcctagcaccagcagaccccccagaccttgggacaccccctgcccccctgtgtacgccgagaccaagcacttcctgtacagcagcggcgacaaggagcagctgagacccagcttcctgctgagctccctgagacccagcctgaccggcgccagaagactggtggagaccatcttcctgggcagcagaccctggatgcccggcacccccagaagactgcccagactgccccagagatactggcagatgagacccctgttcctggagctgctgggcaaccacgcccagtgcccctacggcgtgctgctgaagacccactgccccctgagagctgccgtgacccccgcagctggcgtgtgcgccagagagaagccccagggcagcgtggccgctcccgaggaggaggacaccgatcccagaagactggtgcagctgctgagacagcacagcagcccctggcaggtgtacggcttcgtgagagcctgcctgagaagactggtgcctcccggcctgtggggcagcagacacaacgagagaagattcctgagaaacaccaagaagttcatcagcctgggcaagcacgccaagctgagcctccaggagctgacatggaagatgagcgtgagagactgcgcctggctgaggagaagccctggcgtgggctgcgtgcccgccgccgagcacagactgagagaggagatcctggccaagtttctgcactggctgatgagcgtgtacgtggtggagctgctgagaagcttcttctacgtgaccgagaccacattccagaagaacagactgttcttttacaggaagagcgtgtggagcaagctccagagcatcggcatcagacagcacctgaagagagtgcagctgagagagctgagcgaggccgaggtgagacagcacagagaggccagacccgccctgctgaccagcagactgagattcatccccaagcccgatggcctgagacccatcgtgaacatggactacgtggtgggagccagaacctttagaagagagaagagagccgagagactgaccagcagagtgaaggccctgttcagcgtgctgaactacgagagagccagaagacccggcctgctgggcgccagcgtgctgggcctggacgacatccacagagcctggagaaccttcgtgctgagagtgagagcccaggaccctcctcccgagctgtacttcgtgaaggtggacgtgaccggcgcctacgacaccatcccccaggacagactgaccgaggtgatcgccagcatcatcaagccccagaacacctactgcgtgagaagatacgccgtggtgcagaaggccgcccacggccacgtgagaaaggccttcaagagccacgtgagcaccctgaccgacctccagccctacatgagacagttcgtggcccacctccaggagaccagccccctgagagatgccgtggtgatcgagcagagctcttccctgaacgaggcctccagcggcctgttcgacgtgttcctgagattcatgtgccaccacgccgtgagaatcagaggcaagagctacgtgcagtgccagggcatcccccagggcagcatcctgagcaccctgctgtgcagcctgtgctacggcgacatggagaacaagctgttcgctggcatcagaagagacggcctgctgctgagactggtggacgacttcctgctggtgaccccccacctgacccacgccaagaccttcctgagaaccctggtgagaggcgtgcccgagtacggctgcgtggtgaacctgagaaagaccgtggtgaactttcccgtggaggacgaggccctgggcggcaccgccttcgtgcagatgcccgcccacggcctgtttccctggtgcggcctgctcctcgacaccagaaccctggaggtgcagagcgactacagcagctacgcaagaaccagcatcagagccagcctgaccttcaacagaggcttcaaggccggcagaaacatgagaagaaagctgttcggcgtgctgagactgaagtgccacagcctgttcctggacctccaggtgaacagcctccagaccgtgtgcaccaacatctacaagatcctgctgctccaggcctacagattccacgcctgcgtgctccagctgcccttccaccagcaggtgtggaagaatcccaccttcttcctgagagtgatcagcgacaccgccagcctgtgctacagcatcctgaaggccaagaatgccggcatgagcctgggcgccaagggcgccgctggacccctgcccagcgaggccgtgcagtggctgtgccaccaggccttcctgctgaagctgaccagacacagagtgacctacgtgcccctgctgggcagcctgagaaccgcccagacccagctgagcagaaagctgcctggcacaaccctgaccgccctggaggcagccgcaaaccccgccctgcccagcgacttcaagaccatcctggactag

SEQ ID NO: 11 - protein, 248 aa, Artificial Sequence
  amino acid sequence of tTA
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGGPADALDDFDLDMLPADALDDFDLDMLPADALDDFDLDMLPG

SEQ ID NO: 12 - DNA, 747 bp, Artificial Sequence
  nucleotide sequence of tTA
atgtcaaggctggataaaagcaaagtgattaactccgctctggaactgctgaacgaagtcggcattgaggggctgaccacacgcaagctggcacagaagctgggagtggagcagcccaccctgtactggcacgtgaagaacaagcgcgccctgctggacgccctggccatcgagatgctggatcggcaccacacacacttctgccctctggagggcgagagctggcaggacttcctgcggaacaatgccaagagctttagatgtgccctgctgtcccacagggatggagcaaaggtgcacctgggcaccagaccaacagagaagcagtacgagaccctggagaaccagctggccttcctgtgccagcagggcttttctctggagaatgccctgtatgccctgagcgccgtgggacacttcaccctgggatgcgtgctggaggaccaggagcaccaggtggccaaggaggagagagagacacctaccacagactccatgccccctctgctgaggcaggccatcgagctgtttgatcaccagggcgccgagccagccttcctgtttggcctggagctgatcatctgcggcctggagaagcagctgaagtgtgagtctggaggaccagcagatgccctggacgatttcgacctggatatgctgcccgccgacgccctggacgattttgatctggacatgctgcctgctgatgccctggatgattttgacctggatatgctgcctggataa

SEQ ID NO: 13 - protein, 199 aa, Artificial Sequence
  amino acid sequence of PuroR
MTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 14 - DNA, 600 bp, Artificial Sequence
  nucleotide sequence of PuroR
atgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 15 - protein, 124 aa, Artificial Sequence
  amino acid sequence of ZeoR
MAKLTSAVPVLTARDVAGAVEFWTDRLGFSRDFVEDDFAGVVRDDVTLFISAVQDQVVPDNTLAWVWVRGLDELYAEWSEVVSTNFRDASGPAMTEIGEQPWGREFALRDPAGNCVHFVAEEQD

SEQ ID NO: 16 - DNA, 375 bp, Artificial Sequence
  nucleotide sequence of ZeoR
atggccaagttgaccagtgccgttccggtgctcaccgcgcgcgatgtggccggagcggtcgagttctggaccgaccggctcgggttcagccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactaa

SEQ ID NO: 17 - DNA, 20 bp, Artificial Sequence
  GX535 Forward primer
ctgattgacatggatcacga

SEQ ID NO: 18 - DNA, 20 bp, Artificial Sequence
  GX534 Reverse primer
ctctcgtcgatgaactgctt